SEQUENCE LISTING (10 sequences)

SEQ ID NO: 1 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 2 - protein, 133 aa, Homo sapiens [sequence omitted]

SEQ ID NO: 3 - protein, 252 aa, Homo sapiens
MFFWCACCLMVAWRVSASDAEHCPELPPVDNSIFVAKEVEGQILGTYVCIKGYHLVGKKTLFCNASKEWDNTTTECRLGHCPDPVLVNGEFSSSGPVNVSDKITFMCNDHYILKGSNRSQCLEDHTWAPPFPICKSRDCDPPGNPVHGYFEGNNFTLGSTISYYCEDRYYLVGVQEQQCVDGEWSSALPVCKLIQEAPKPECEKALLAFQESKNLCEAMENFMQQLKESGMTMEELKYSLELKKAELKAKLL

SEQ ID NO: 4 - protein, 59 aa, Homo sapiens
IQEAPKPECEKALLAFQESKNLCEAMENFMQQLKESGMTMEELKYSLELKKAELKAKLL

SEQ ID NO: 5 - protein, 116 aa, Homo sapiens
RDCDPPGNPVHGYFEGNNFTLGSTISYYCEDRYYLVGVQEQQCVDGEWSSALPVCKLIQEAPKPECEKALLAFQESKNLCEAMENFMQQLKESGMTMEELKYSLELKKAELKAKLL

SEQ ID NO: 6 - protein, 227 aa, Artificial Sequence
  Hi2cb fusion protein
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSIQEAPKPECEKALLAFQESKNLCEAMENFMQQLKESGMTMEELKYSLELKKAELKAKLL

SEQ ID NO: 7 - protein, 227 aa, Artificial Sequence
  Hi2mcb fusion protein
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISRINVIVLELKGSETTFMCEYADETATIVEFLNRWITFCQSIISTLTGGGGSGGGGSGGGGSIQEAPKPECEKALLAFQESKNLCEAMENFMQQLKESGMTMEELKYSLELKKAELKAKLL

SEQ ID NO: 8 - protein, 5 aa, Artificial Sequence
  linker repeat
GGGGS

SEQ ID NO: 9 - protein, 207 aa, Artificial Sequence
  Hi2cb mature
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSIQEAPKPECEKALLAFQESKNLCEAMENFMQQLKESGMTMEELKYSLELKKAELKAKLL

SEQ ID NO: 10 - protein, 207 aa, Artificial Sequence
  Hi2mcb mature
APTSSSTKKTQLQLEHLLLDLQMILNGINNYKNPKLTRMLTFKFYMPKKATELKHLQCLEEELKPLEEVLNLAQSKNFHLRPRDLISRINVIVLELKGSETTFMCEYADETATIVEFLNRWITFCQSIISTLTGGGGSGGGGSGGGGSIQEAPKPECEKALLAFQESKNLCEAMENFMQQLKESGMTMEELKYSLELKKAELKAKLL

(19) United States
(12) Patent Application Publication
(10) Pub. No.: US 2023/0036793 A1
(43) Pub. Date: Feb. 2, 2023

(54) INTERLEUKIN 2 CHIMERIC CONSTRUCTS

(71) Applicants: ILTOO PHARMA, Paris (FR); SORBONNE UNIVERSITE, Paris (FR); INSERM (INSTITUT NATIONAL DE LA SANTÉ ET DE LA RECHERCHE MEDICALE), Paris (FR); ASSISTANCE PUBLIQUE - HÔPITAUX DE PARIS, Paris (FR)

(72) Inventors: David KLATZMANN, Paris (FR); Nicolas BILLIALD, Paris (FR); Thomas VAZQUEZ, Montreuil (FR); Jérémie MARIAU, Fontenay sous Bois (FR)

(21) Appl. No.: 17/784,492

(22) PCT Filed: Dec. 11, 2020
(86) PCT No.: PCT/EP2020/085830

§ 371 Date: Jun. 10, 2022

(30) Foreign Application Priority Data
Dec. 12, 2019 (EP) 19306637.0

(51) Int. Cl. C07K 14/55; C07K 14/47; A61P 37/06

(52) U.S. Cl. CPC C07K 14/55; C07K 14/4703; A61P 37/06; C07K 2319/00; A61K 38/00

(57) ABSTRACT

The present invention relates to a chimeric construct, comprising i) an interleukin 2 (IL2) moiety and ii) a beta chain of the C4b-binding protein (C4BPβ) or at least one fragment or functional variant thereof that is capable of forming a dimeric protein.

The present invention relates to IL2 constructs with improved pharmacokinetics and/or pharmacodynamics.

### BACKGROUND OF THE INVENTION

Interleukin-2 (IL2 or IL-2) is a cytokine that regulates key aspects of the immune system. IL2 has been used in attempts to boost immune responses in patients with cancer, as well as autoimmune and/or inflammatory diseases. IL2 is a potent T cells growth factor that promotes immune responses, including clonal expansion of antigen-activated T cells, drives development of CD4+T-helper (Th)I and Th2 cells, terminally differentiates CD8+ cytotoxic T lymphocytes (CTLs), and opposes development of CD4+ThI7 and T-follicular helper (Tfh) cells. IL2 also shapes T cell memory recall responses.

Low doses of IL2 have been used to selectively boost tolerance to suppress unwanted immune responses associated with autoimmune-like attack of self tissues. The experience thus far has been that this therapy is safe, with no indication of reactivation of auto-aggressive T cells, while regulatory T cells (Tregs) increase in nearly all patients, which is accompanied by clinical improvement.

Nevertheless, IL2 as a therapeutic can be improved, notably regarding its short-half life in vivo. For these reasons new IL2 biologics are needed having improved pharmacokinetics and/or pharmacodynamics.

### SUMMARY OF THE INVENTION

The invention provides a chimeric construct comprising i) an interleukin 2 (IL2) moiety and ii) a beta chain of the C4b-binding protein (C4BPβ) or at least one fragment or functional variant thereof that is capable of forming a dimeric protein.

Such constructs, hence the IL2 moiety, have an improved half-life. Furthermore the inventors have surprisingly shown that such chimeric constructs improve the selectivity of Treg expansion.

In a particular embodiment, the chimeric construct is in dimer form, wherein the monomers are associated by covalent bonding between two cysteines of C4BPβ. Homodimers and heterodimers are described in greater details below.

In a preferred embodiment, the C4BPβ fragment comprises, or consists of, amino acid residues 194 to 252 of C4BPβ, or a longer fragment of C4BPβ that extends at the N-term up to at most amino acid 135.

In a preferred embodiment, said II-2 moiety is human IL-2 or homologous variant thereof, wherein the variant has at least 85% amino acid identity with human wild-type IL-2, preferably wherein the variant is an active analogue of human IL-2 which has at least 90% amino acid identity with human wild-type IL-2, wherein said IL-2 moiety is preferably an IL2 mutein that comprises a substitution at position N88 of SEQ ID NO: 2, still preferably substitution N88R.

### LEGENDS TO THE FIGURES

FIG. 1 shows the dose-response of pSTAT5 induction in Treg (A), Tconv (B) and CD8 T cells (C). Hi2 is human IL-2 (SEQ ID NO:1), Hi2cb is human IL-2 fused to the C-terminal region of C4BPß (SEQ ID NO:6), Hi2mcb is a mutated IL-2 fused to the C-terminal region of C4BPß (SEQ ID NO: 7)

FIG. 2 shows kinetic curves of the fold increase of four different T cell compartments and NK cells in mice after injection of 10¹¹ viral genome of AAV expressing IL-2 constructs.

FIG. 3 shows the kinetic of plasma (A) and urinary (B) human IL-2 over the time, in mice after injection of 10¹¹ viral genome of AAV expressing IL-2 constructs.

FIG. 4 shows kinetic curves of the fold increase of four different T cell compartments and NK cells in mice after injection of 10¹² viral genome of AAV expressing IL-2 constructs.

FIG. 5 shows the survival Kaplan-Meier curve of mice after injection of 10¹² viral genome of AAV expressing IL-2 constructs.

FIG. 6 shows the therapeutic efficacy of fusion proteins Hi2cb or Hi2mcb, compared to Hi2 in a model of experimental autoimmune encephalomyelitis (EAE).

FIG. 7 shows the experimental administration schedule based on one injection of 25 000 International Units of Hi2, Hi2cb or Hi2mcb every day during five days. Immunophenotyping was carried out every day before injection to assess Treg kinetic.

FIG. 8 shows the pharmacokinetic profile of each construction Hi2, Hi2cb or Hi2mcb overtime after a single subcutaneous injection.

### DETAILED DESCRIPTION OF THE INVENTION

### Definitions

The “subject” or “patient” to be treated may be any mammal, preferably a human being. The human subject may be a child, an adult or an elder.

The term “treating” or “treatment” means any improvement in the disease. It includes alleviating at least one symptom, or reducing the severity or the development of the disease. When the disease is an inflammatory and/or autoimmune disorder, the term more particularly includes reducing the risk, occurrence or severity of acute episodes (flares). The term “treating” or “treatment” encompasses reducing the progression of the disease. In particular the invention encompasses preventing or slowing down the progression of the disease. The term “treating” or “treatment” further encompasses prophylactic treatment, by reducing the risk or delaying the onset of the disease, especially in a subject who is asymptomatic but has been diagnosed as being “at risk”.

“Regulatory T cells” or “Tregs” are T lymphocytes having immunosuppressive activity. Natural Tregs are characterized as CD4+CD25+Foxp3+ cells. Tregs play a major role in the control of inflammatory diseases, although their mode of action in such disease is not well understood. In fact, in most inflammatory diseases, Treg depletion exacerbates disease while Treg addition decreases it. Most Tregs are CD4+ cells, although there also exists a rare population of CD8+Foxp3+T lymphocytes with a suppressive activity.

Within the context of this application, “effector T cells” (or “Teff”) designates conventional T lymphocytes other than Tregs (sometimes also referred to as Tconv in the literature), which express one or more T cell receptor (TCR) and perform effector functions (e.g., cytotoxic activity, cytokine secretion, etc). Major populations of human Teff according to this invention include CD4+T helper lymphocytes (e.g., Th0, Th1, Th2, Th9, Th17, Tfh) and CD4+ or CD8+ cytotoxic T lymphocytes, and they can be specific for self or non-self antigens. Teff does not comprise the Foxp3+ regulatory CD8+ T cells.

Within the context of this application, “T follicular helper cells” (or “Tfh”) designates T CD4+ lymphocytes that express BcL6, CXCR5 and PD1, are Foxp3−, and provide B cell help.

Within the context of this application, “T follicular regulatory cells” (or “Tfr”) designates CD4+ CXCR5+PD−1+Bcl6+Foxp3+CD25− T lymphocytes.

The sequence listing shows the following sequences:

SEQ ID NO: 1 is wild-type human IL2 (253 amino acids, including the signal peptide)

SEQ ID NO: 2 is mature wild-type human IL2 (233 amino acids, including the signal peptide)

SEQ ID NO: 3 is C4BP beta chain (1-252)

SEQ ID NO: 4 is fragment 194-252 of C4BP beta chain

SEQ ID NO: 5 is fragment 137-252 of C4BP beta chain

SEQ ID NO: 6 is the amino acid sequence of Hi2cb (including the signal peptide)

SEQ ID NO: 7 is the amino acid sequence Hi2mcb (N88R), including the signal peptide

SEQ ID NO: 8 is the GGGGS pattern (linker)

SEQ ID NO: 9 is the amino acid sequence of Hi2cb without the signal peptide

SEQ ID NO: 10 is the amino acid sequence Hi2mcb (N88R) without the signal peptide

The IL-2 Moiety

As used herein, Interleukin-2 (IL-2) encompasses mammal wild type Interleukin-2, and variants thereof. Preferably, IL-2 is a human IL-2, or a variant thereof.

Active variants of IL-2 have been disclosed in the literature. Variants of the native IL-2 can be fragments, analogues, and derivatives thereof. By “fragment” is intended a polypeptide comprising only a part of the polypeptide sequence. An “analogue” designates a polypeptide comprising the native polypeptide sequence with one or more amino acid substitutions, insertions, or deletions. Muteins and pseudopeptides are specific examples of analogues. “Derivatives” include any modified native IL-2 polypeptide or fragment or analogue thereof, such as glycosylated, phosphorylated, fused to another polypeptide or molecule, polymerized, etc., or through chemical or enzymatic modification or addition to improve the properties of IL-2 (e.g., stability, specificity, etc.). The IL-2 moiety of active variants generally has at least 75%, preferably at least 80%, 85%, more preferably at least 90% or at least 95% amino acid sequence identity to the amino acid sequence of the reference IL-2 polypeptide, for instance mature wild type human IL-2.

As used herein, “wild type IL-2” means IL-2, whether native or recombinant, comprising the 133 normally occurring amino acid sequence of native human IL-2, whose amino acid sequence is described in Fujita, et. al., PNAS USA, 80,7437-7441 (1983). SEQ ID NO: 2 (133 amino acids) is the human IL-2 sequence less the signal peptide, consisting of an additional 20 N-terminal amino acids. SEQ ID NO:1 (153 amino acids) is the human IL-2 sequence including the signal peptide.

As used herein, “IL-2 mutein” means a polypeptide in which specific amino acid substitutions to the human mature interleukin-2 protein have been made. All numbering of the amino acids is made with respect to human mature interleukin-2 protein of SEQ ID NO: 2, unless otherwise indicated.

In some embodiments, the cysteine at position 125 is replaced with a neutral amino acid such as serine (C125S), alanine (C125A), threonine (C125T) or valine (C125V).

For example, elimination of the O-glycosylation site results in a more homogenous product when active variant is expressed in mammalian cells such as CHO or HEK cells.

In certain embodiments active variant comprises an additional amino acid mutation which eliminates the O-glycosylation site of IL-2 at a position corresponding to residue 3 of human IL-2. In one embodiment said additional amino acid mutation which eliminates the 0-glycosylation site of IL-2 at a position corresponding to residue 3 of human IL-2 is an amino acid substitution. Exemplary amino acid substitutions include T3A, T3G, T3Q, T3E, T3N, T3D, T3R, T3K, and T3P. In a specific embodiment, said additional amino acid mutation is the amino acid substitution T3A.
Active variants that selectively promote T-reg cell proliferation, survival, activation and/or function are particularly useful in treating inflammatory and/or autoimmune disorders.

By “selectively promote,” it is meant that the active variant promotes the activity in T-reg cells but has limited or lacks the ability to promote the activity in non-regulatory T cells. Further described herein are assays to screen for active variants that selectively promote T-reg cell proliferation, survival, activation and/or function.

Methods for determining whether a variant IL-2 polypeptide is active are available in the art. See e.g. WO2016/014428. An active variant is defined as a variant that shows an ability to stimulate Tregs, including variants with an improved ability, or a similar ability, or even a reduced ability to stimulate Tregs when compared to wild-type IL-2 or aldesleukin (as defined below), to the extent it does not stimulate Teffs more than it stimulates Tregs. Methods for testing whether a candidate molecule stimulate T cells, Tregs in particular, or NK cells are well-known. Variants may be tested for their ability to stimulate effector T cells (such as CD8+ T cells), CD4+Foxp3+ Tregs, or NK cells. In a preferred embodiment, the active variant shows a reduced ability to stimulate NK cells, compared to wild type IL2 or aldesleukin. Monitoring STAT5 phosphorylation is a simple way of assessing variants for their ability to preferentially stimulate Tregs over Teff, as described in Yu et al, Diabetes 2015; 64:2172-2183. In a particular embodiment, a variant is particularly useful when a given level of STAT5 phosphorylation is achieved with doses at least 10 times inferior for Tregs than for other immune cells, including Teffs.

Said active variants induce signaling events that preferentially induce survival, proliferation, activation and/or function of Treg cells. In certain embodiments, the IL-2 variant retains the capacity to stimulate, in Treg cells, STAT5 phosphorylation and/or phosphorylation of one or more of signaling molecules downstream of the IL-2R, e.g., p38, ERK, SYK and LCK. In other embodiments, the IL-2 variant retains the capacity to stimulate, in Treg cells, transcription or protein expression of genes or proteins, such as FOXP3, Bcl-2, CD25 or IL-10, that are important for Treg cell survival, proliferation, activation and/or function. In other embodiments, the IL-2 variant exhibits a reduced capacity to stimulate endocytosis of IL-2/IL-2R complexes on the surface of CD25+ T cells. In other embodiments, the IL-2 variant demonstrates inefficient, reduced, or absence of stimulation of PI3-kinase signaling, such as inefficient, reduced or absent phosphorylation of AKT and/or mTOR (mammalian target of rapamycin). In yet other embodiments, the IL-2 variant retains the ability of wild type IL-2 to stimulate STAT5 phosphorylation and/or phosphorylation of one or more of signaling molecules downstream of the IL-2R in Treg cells, yet demonstrates inefficient, reduced, or absent phosphorylation of STAT5, AKT and/or mTOR or other signaling molecules downstream of the IL-2R in FOXP3− CD4+ or CD8+ T cells or NK cells. In other embodiments, the IL-2 variant is inefficient or incapable of stimulating survival, growth, activation and/or function of FOXP3− CD4+ or CD8+ T cells or NK cells.

In all cases, these variants have the capacity to stimulate cell lines such as CTLL-2 or HT-2 which can be universally used to determine their biological activity.

For instance, the biological activity of IL-2 may be determined by a cell-based assay performed on HT-2 cell line (clone A5E, ATCC® CRL-1841™) whose growth is dependent on IL-2. Cell growth in the presence of a range of test interleukin-2 product is compared with the growth recorded with IL-2 international standard (WHO 2nd International Standard for INTERLEUKIN 2 (Human, rDNA derived) NIBSC code: 86/500). Cell growth is measured after addition and transformation of [3-(4,5-dimethylthiazol-2-yl)-5-(3-carboxymethoxyphenyl)-2-(4-sulfophenyl)-2H-tetrazolium (inner salt, MTS) into formazan by active viable cells. Formazan concentration is then measured by spectrophotometry at 490 nm.

Examples of IL-2 variants are disclosed, for instance, in EP109748, EP136489, U.S. Pat. No. 4,752,585; EP200280, EP118617, WO99/60128, EP2288372, U.S. Pat. Nos. 9,616,105, 9,580,486, WO2010/085495, WO2016/164937.

For instance, certain mutations may result in a reduced affinity for the signaling chains of the IL-2 receptor (IL-2Rβ/CD122 and/or IL-2Rγ/CD132) and/or a reduced capacity to induce a signaling event from one or both subunits of the IL-2 receptor. Other mutations may confer higher affinity for CD25 (IL-2Rα). In both cases, those mutations define active variants that preferentially induce survival, proliferation, activation and/or function of Treg. This property may be monitored using surface plasmon resonance.

Particular examples of useful variants include IL-2 muteins which show at least one amino acid substitution at position D20, N30, Y31, K35, V69, Q74, N88, V91, or Q126, numbered in accordance with wild type IL-2, meaning that the chosen amino acid is identified with reference to the position at which that amino acid normally occurs in the mature sequence of wild type IL-2 of SEQ ID NO:2.

Preferred IL-2 muteins comprise at least one substitution at position D20H, D20I, D20Y, N30S, Y31H, K35R, V69AP, Q74, N88R, N88D, N88G, N881, V91K, or Q126L.

In some embodiments, the IL-2 mutein molecule comprises a V91K substitution. In some embodiments, the IL-2 mutein molecule comprises a N88D substitution. In some embodiments, the IL-2 mutein molecule comprises a N88R substitution. In some embodiments, the IL-2 mutein molecule comprises a substitution of H16E, D84K, V91N, N88D, V91K, or V91R, any combinations thereof. In some embodiments, these IL-2 mutein molecules also comprise a substitution at position 125 as described herein. In some embodiments, the IL-2 mutein molecule comprises one or more substitutions selected from the group consisting of: T3N, T3A, L12G, L12K, L12Q, L 12S, Q13G, E15A, E15G, E15S, H16A, H16D, H16G, H16K, H16M, H16N, H16R, H16S, H16T, H16V, H16Y, L19A, L19D, L19E, L19G, L19N, L19R, L19S, L19T, L19V, D20A, D20E, D20H, D20I, D20Y, D20F, D20G, D20T, D20W, M23R, R81A, R81G, R81 S, R81T, D84A, D84E, D84G, D841, D84M, D84Q D84R, D84S, D84T, S87R, N88A, N88D, N88E, N881, N88F, N88G, N88M, N88R, N88S, N88V, N88W, V91D, V91E, V91G, V91 S, 192K, 192R, E95G, and Q126. In some embodiments, the amino acid sequence of the IL-2 mutein molecule differs from the amino acid sequence set forth in mature IL-2 sequence with a C125A or C125S substitution and with one substitution selected from T3N, T3A, L12G, L12K, L12Q L12S, Q13G, E15A, E15G, E15S, H16A, H16D, H16G, H16K, H16M, H16N, H16R, H16S, H16T, H16V, H16Y, L19A, L19D, L19E, L19G, L19N, L19R, L19S, L19T, L19V, D20A, D20E, D20F, D20G, D20T, D20W, M23R, R81A, R81G, R81 S, R81T, D84A, D84E, D84G, D841, D84M, D84Q, D84R, D84S, D84T, S87R, N88A, N88D, N88E, N88F, N881, N88G, N88M, N88R, N88S, N88V, N88W, V91D, V91E, V91G, V91 S, 192K, 192R, E95G, Q1261, Q126L, and Q126F. In some embodiments, the IL-2 mutein molecule differs from the amino acid sequence set forth in mature IL-2 sequence with a C125A or C125S substitution and with one substitution selected from D20H, D20I, D20Y, D20E, D20G, D20W, D84A, D84S, H16D, H16G, H16K, H16R, H16T, H16V, 192K, 192R, L12K, L19D, L19N, L19T, N88D, N88R, N88S, V91D, V91G, V91K, and V91S. In some embodiments, the IL-2 mutein comprises N88R and/or D20H mutations.

These substitutions can be used alone or in combination with one another. In some embodiments, the mutein comprises each of these substitutions. In some embodiments, the mutein comprises 1, 2, 3, 4, 5, 6, 7, or 8 of these mutations.

In some embodiments, the IL-2 mutein comprises a N88R or a N88D mutation, preferably N88R. In some embodiments, the IL-2 mutein comprises a C125A or C125S mutation. These substitutions can be used alone or in combination with one another. In some embodiments, the mutein comprises 1, 2, 3, 4, 5, 6, 7, or 8 of these mutations. In some embodiments, the mutein comprises each of these substitutions.

In a particular embodiment, the IL-2 moiety is aldesleukin. Aldesleukin is the active ingredient of Proleukin®. Aldesleukin is a variant of mature human IL-2 comprising two amino acid modifications as compared to the sequence of mature human IL-2 (SEQ ID NO:2): the deletion of the first amino acid (alanine) and the substitution of cysteine at position 125 by serine.

Conservative modifications and substitutions at other positions of IL-2 (i. e., those that have a minimal effect on the secondary or tertiary structure of the mutein) are encompassed. Such conservative substitutions include those described by Dayhoff in The Atlas of Protein Sequence and Structure 5 (1978), and by Argos in EMBO J., 8: 779-785 (1989). For example, amino acids belonging to one of the following groups represent conservative changes: -ala, pro, gly, gln, asn, ser, thr; -cys, ser, tyr, thr; -val, ile, leu, met, ala, phe; -lys, arg, his; -phe, tyr, trp, his; and -asp, glu.

Variants with mutations which disrupt the binding to the a subunit of IL-2R are not preferred, as those mutants may have a reduced capacity to stimulate Tregs.
Active variants that promote Teff cell proliferation, survival, activation and/or function may be useful in treating cancers.

Such active variants of IL-2 comprise at least one amino acid mutation that abolishes or reduces affinity of the mutant IL-2 polypeptide to the α-subunit of the IL-2 receptor (CD25) and preserves affinity of the mutant IL-2 polypeptide to the intermediate-affinity IL-2 receptor, each compared to a wild-type IL-2 polypeptide. This property may be monitored using surface plasmon resonance.

Preferred active variants include IL-2 mutein comprising F42A, K43N, Y45A, and/or E62A substitution(s).

Active variants such as mutants of human IL-2 (hIL-2) with decreased affinity to CD25 may for example be generated by amino acid substitution at amino acid position 35, 38, 42, 43, 45, 62 or 72 or combinations thereof (numbering relative to the human IL-2 sequence SEQ ID NO: 2). Exemplary amino acid substitutions include K35E, K35A, R38A, R38E, R38N, R38F, R38S, R38L, R38G, R38Y, R38W, F42L, F42A, F42G, F42S, F42T, F42Q, F42E, F42N, F42D, F42R, F42K, K43E, Y45A, Y45G, Y45S, Y45T, Y45Q, Y45E, Y45N, Y45D, Y45R, Y45K, E62G, E62A, E62S, E62T, E62Q, E62E, E62N, E62D, E62R, E62K, L72G, L72A, L72S, L72T, L72Q, L72E, L72N, L72D, L72R, and L72K. Particular active variants useful in the chimeric construct for the present invention comprise an amino acid mutation at an amino acid position corresponding to residue 42, 45, or 72 of human IL-2, or a combination thereof. In one embodiment said amino acid mutation is an amino acid substitution selected from the group of F42A, F42G, F42S, F42T, F42Q, F42E, F42N, F42D, F42R, F42K, Y45A, Y45G, Y45S, Y45T, Y45Q, Y45E, Y45N, Y45D, Y45R, Y45K, L72G, L72A, L72S, L72T, L72Q, L72E, L72N, L72D, L72R, and L72K, more specifically an amino acid substitution selected from the group of F42A, Y45A and L72G. These active variants exhibit substantially similar binding affinity to the intermediate-affinity IL-2 receptor, and have substantially reduced affinity to the α-subunit of the IL-2 receptor and the high-affinity IL-2 receptor (IL2Rαβγ) compared to a wild-type form of the IL-2 mutant.

Other characteristics of useful active variants may include the ability to induce proliferation of IL-2 receptor-bearing T and/or NK cells, the ability to induce IL-2 signaling in IL-2 receptor-bearing T and/or NK cells, the ability to generate interferon (IFN)-y as a secondary cytokine by NK cells, a reduced ability to induce elaboration of secondary cytokines—particularly IL-10 and TNF-a—by peripheral blood mononuclear cells (PBMCs), a reduced ability to activate regulatory T cells, a reduced ability to induce apoptosis in T cells, and a reduced toxicity profile in vivo.

Particular active variants comprise three amino acid mutations that abolish or reduce affinity of the active variants to the α-subunit of the IL-2 receptor but preserve affinity of the active variant to the intermediate affinity IL-2 receptor. In one embodiment said three amino acid mutations are at positions corresponding to residue 42, 45 and 72 of human IL-2. In one embodiment said three amino acid mutations are amino acid substitutions. In one embodiment said three amino acid mutations are amino acid substitutions selected from the group of F42A, F42G, F42S, F42T, F42Q, F42E, F42N, F42D, F42R, F42K, Y45A, Y45G, Y45S, Y45T, Y45Q, Y45E, Y45N, Y45D, Y45R, Y45K, L72G, L72A, L72S, L72T, L72Q, L72E, L72N, L72D, L72R, and L72K. In a specific embodiment said three amino acid mutations are amino acid substitutions F42A, Y45A and L72G (numbering relative to the human IL-2 sequence of SEQ ID NO: 2).

In certain embodiments said amino acid mutation reduces the affinity of the mutant IL-2 polypeptide to the α-subunit of the IL-2 receptor by at least 5-fold, specifically at least 10-fold, more specifically at least 25-fold. In embodiments where there is more than one amino acid mutation that reduces the affinity of the active variant to the α-subunit of the IL-2 receptor, the combination of these amino acid mutations may reduce the affinity of the active variant to the α-subunit of the IL-2 receptor by at least 30-fold, at least 50-fold, or even at least 100-fold. In one embodiment said amino acid mutation or combination of amino acid mutations abolishes the affinity of the active variant to the α-subunit of the IL-2 receptor so that no binding is detectable by surface plasmon resonance.

Substantially similar binding to the intermediate-affinity receptor, i.e. preservation of the affinity of the mutant IL-2 polypeptide to said receptor, is achieved when the active variant exhibits greater than about 70 percent of the affinity of a wild-type form of the IL-2 mutant to the intermediate-affinity IL-2 receptor. Active variants useful in the invention may exhibit greater than about 80 percent and even greater than about 90 percent of such affinity.

Reduction of the affinity of IL-2 for the α-subunit of the IL-2 receptor in combination with elimination of the O-glycosylation of IL-2 results in an IL-2 protein with improved properties.

In a specific embodiment, the active variant can elicit one or more of the cellular responses selected from the group consisting of: proliferation in an activated T lymphocyte cell, differentiation in an activated T lymphocyte cell, cytotoxic T cell (CTL) activity, proliferation in an activated B cell, differentiation in an activated B cell, proliferation in a natural killer (NK) cell, cytotoxic activity in a NK cell, differentiation in a NK cell, cytokine secretion by an activated T cell or an NK cell, and NK/lymphocyte activated killer (LAK) antitumor cytotoxicity.

In some embodiments, these active variants also comprise a substitution at position 125 as described herein.

The C4BPβ or C4BPβ Fragment

The C4BP protein is involved in coagulation and the complement system. The major form of C4BP is composed of 7 identical 75 kD alpha chains and one 45 kD beta chain. The alpha and beta chains respectively contain 8 and 3 SCR (short consensus repeat) domains, those motifs being found in many complement-regulating proteins and constituted by 50-70 amino acids organized into beta sheets. The amino acid sequence of the beta chain of human C4BP is shown as SEQ ID NO:3.

A nucleic acid sequence corresponding to this polypeptide sequence has also been described by Hillarp and Dahlback (1990, PNAS, vol 87, pp 1183-1187).

The role of the alpha chain in polymerizing the C4BP protein has been studied by Kask et al (Biochemistry 2002, 41, 9349-9357). Those authors have shown that the C-terminal portion of the alpha chain, in particular its a helical structure and the presence of two cysteines, is necessary for polymerization of the C4BP protein when the alpha chain is expressed in a heterologous system.

European patent application 2 227 030 describes the production of heteromultimeric recombinant proteins by using C-terminal fragments of the alpha and beta chains of the C4BP protein in fusion with polypeptides of interest.

U.S. Pat. No. 7,884,190 describes the use of the beta chain of the C4BP protein, independently of its use in association with the alpha chain of the C4BP protein for the production of dimeric proteins.

The C4BP protein used to carry out the invention is advantageously the human C4BP protein.

In a preferred embodiment, the IL2 moiety is fused to a fragment of the C4BP β chain that comprises or consists of at least amino acids 194 to 252 (SEQ ID NO: 4).

Sequences coding for longer fragments of the beta chain, or even the whole beta chain, may also be used. For certain applications, it is preferable to avoid using a sequence coding for a beta chain which is capable of binding the S protein participating in coagulation. If the selected sequence codes for a fragment containing the two first SCR motifs of the beta chain, these will preferably by versions mutated by addition, deletion or substitution of amino acids to cut out with the possibility of interaction with the S protein. SCR motifs and/or [GS] domains may be added with the aim of modifying, for example increasing, the flexibility of the fusion polypeptide obtained or to allow the chimeric protein to adopt a suitable conformation to form multimers, particularly dimers.

A longer fragment of C4BPβ that extends at the N-term up to at most amino acid 135 may be used.

In a particular embodiment, the fragment of the C4BP β chain may comprise or consist of at least amino acids 185 to 252, 180 to 252, 175 to 252, 170 to 252, 165 to 252, 160 to 252, 155 to 252, 150 to 252, 145 to 252, 140 to 252, or 135 to 252 (with respect to SEQ ID NO:3).

In a particular embodiment, the fragment of the C4BP β chain comprises or consists of at least amino acids 137 to 252 (SEQ ID NO: 5).

A functional variant of C4BPβ may be used. The functional variant has maintained the capacity to form at least one dimer, for example a homodimer or a heterodimer, a trimer, a tetramer or any multimer containing a different number of chimeric proteins.

Within the context of the invention, the term “functional variant of a fragment of the C4BP β chain” means a polypeptide sequence modified with respect to the sequence of fragment of the beta chain by deletion, substitution or addition of one or more amino acids, said modified sequence retaining, however, the capacity to form at least dimer proteins using the method of the invention. More precisely, the production of dimer proteins using a sequence coding for a functional variant of the fragment may be at least 80% equal to that obtained with a native sequence coding for the fragment (SEQ ID NO: 3, or a fragment thereof), preferably at least 90%, still preferably 95%) in an identical expression system. Preferably, the variant is such that more than 80% of the fusion polypeptides which it contains are produced in the form of dimers in a eukaryotic expression system in accordance with the invention.

In a particular embodiment, a variant of the fragment of the beta chain is encoded by a nucleic acid that is capable of hybridizing under stringent conditions with the wildtype sequence coding for the fragment, as described by Hillarp and Dahlback (1990, PNAS, Vol. 87, pp 1183-1187).

The term “stringent conditions” means conditions which allow specific hybridization of two single strand DNA sequences at about 65° C., for example, in a solution of 6*SSC, 0.5% SDS, 5* Denhardt's solution and 100 μg of non specific DNA or any other solution with an equivalent ionic strength and after washing at 65° C., for example in a solution of at most 0.2*SSC and 0.1% SDS or any other solution with an equivalent ionic strength.

Preferably, the nucleotide sequence coding for a functional variant of said wildtype fragment and hybridizing under stringent conditions with the sequence coding for said fragment has, in the portion which hybridizations, a length of at least 50%, preferably at least 80%, of the length of the sequence coding for the fragment. In a particular implementation, the nucleotide sequence coding for a functional variant of said fragment and hybridizing under stringent conditions with the sequence coding for said fragment has, in the portion which hybridizations, substantially the same length as the sequence coding for said fragment.

In a further implementation, a functional variant is a modified sequence of the wildtype fragment one or more amino acids of which, not essential to the dimerization function, have been removed or substituted and/or one or more amino acids essential to dimerization have been replaced by amino acids with equivalent functional groups (conservative substitution). It is particularly recommended that the two cysteines, located at positions 201 and 215, and the peptide structure around these cysteines be conserved to allow the formation of disulfide bridges which are necessary for dimerization, for example by conservation of at least 3 amino acids upstream and downstream of each cysteine. In particular, a functional variant may also be obtained by inserting a heterologous sequence of the beta chain, and in particular domains of the alpha chain of C4BP, between the cysteines responsible for dimerization or, in contrast, by doing away with certain amino acids present between those same cysteines. Alternatively, a functional variant may be produced by point modification of certain amino acids, in particular substitution of a cysteine responsible for dimerization by a neutral amino acid as regards implication in the dimerization process (for example the amino acids A, V, F, P, M, I, L and W) and at the same time substituting another amino acid by a cysteine to conserve the capacity to form intracatenary and/or intercatenary disulfide bridges between the cysteines. These modifications thus result in a variation in the distance between the various cysteines involved in the multimerization process, in particular dimerization. Preferably, less than 50% of the amino acids of the 194 to 252 fragment are done away with or replaced, preferably less than 25% or even less than 10% (for example 5 amino acids or fewer) or less than 5% (e.g. 1 or 2 amino acids).

In a particular embodiment, the functional variant comprises or consists of
a) a modified sequence of the fragment (preferably the 194-252 fragment) of C4BPβ, wherein less than 25 percent of the amino acids of the fragment (preferably the 194-252 fragment), preferably less than 10 percent, have been cut out or replaced, in which the cysteines located in positions 202 and 216 (numbered with respect to SEQ ID NO: 3) as well as at least 3 amino acids upstream and downstream of each cysteine have been conserved; or
b) a modified sequence of the fragment (preferably the 194-252 fragment) of the C4BPβ, wherein a cysteine responsible for dimerization is substituted with an amino acid, preferably selected from alanine, valine, phenylalanine, proline, methionine, isoleucine, leucine and tryptophan, and another amino acid of the fragment is substituted with a cysteine; or
c) a sequence of the fragment (preferably the 194-252 fragment) of C4BPβ modified by insertion of a sequence which is heterologous to the beta chain, between the cysteines responsible for dimerization; or
d) a sequence of the fragment (preferably the 194-252 fragment) of C4BPβ modified by cutting out amino acids between the cysteines responsible for dimerization.

The Chimeric Constructs

Preferably the IL2 moiety is fused at the N-terminus of C4BPβ or said fragment thereof.

In a preferred embodiment, the chimeric construct comprises a fusion protein wherein one IL2 moiety is fused at the N-term of C4BPβ or of said fragment thereof, and another IL2 moiety is fused at the C-term of C4BPβ or of said fragment thereof. According to such embodiment, the fusion protein comprises the following sequence, from N- to C-term: IL2-C4BPβ-IL2.

The IL2 moiety and C4BPβ or said fragment thereof may be fused in frame (directly) or through an amino acid linker, preferably a polyG linker.

The term “linker” refers to a (poly)peptide comprising 5 to 80 amino acids, preferably 5 to 30, still preferably 10 to 20 amino acids. Suitable linkers are known in the art. In some embodiments, the linker comprises GGGGS (SEQ ID NO: 8) repeats, although an artisan skilled in the art will recognize that other sequences following the general recommendations (Argos, 1990, J Mol Biol. 20; 211(4):943-58; George R, Heringa J. An analysis of protein domain linkers: their classification and role in protein folding. Protein Eng. 2002; 15:871-879) can also be used. Linkers composed of small, non-polar (e.g. Gly) or polar (e.g. Ser or Thr) amino acids provide flexibility, and allows for mobility of the connecting functional domains. In a particular embodiment, the chimeric construct comprises, or consists of; SEQ ID NO:9 or SEQ ID NO:10. Such chimeric construct preferably forms a homodimer, or may be used to produce a heterodimer, as described below.

Homodimer and Heterodimer Constructs

It is herein described a method for producing a recombinant dimer protein comprising:

a) transfecting host cells with a vector allowing expression of a nucleotide sequence coding for a chimeric construct that is a fusion polypeptide comprising i) at least one interleukin 2 (IL2) moiety and ii) a beta chain of the C4b-binding protein (C4BPβ) or at least one fragment or functional variant thereof that is capable of forming a dimeric protein;

b) culturing transfected cells under conditions which are suitable for expressing the nucleotide sequence coding for the fusion polypeptide and the covalent association of two fusion polypeptides in vivo to form a dimeric protein;

c) recovering, and preferably purifying, the dimeric proteins formed.

The transfected cells preferably do not contain any nucleic acid allowing expression of a nucleotide sequence coding for the C-terminal fragment of the alpha chain of the C4BP protein involved in polymerization of the C4BP protein.

In a particular embodiment, it is herein described a method for producing heterodimers, said method comprising:

a. transfecting host cells with one or more vectors to allow the expression of one or more nucleotide sequences coding for:

i. a first fusion polypeptide comprising i) at least one interleukin 2 (IL2) moiety and ii) a beta chain of the C4b-binding protein (C4BPβ) or at least one fragment or functional variant thereof that is capable of forming a dimeric protein; and

ii. a second fusion polypeptide, comprising i) at least one heterologous polypeptide and ii) a beta chain of the C4b-binding protein (C4BPβ) or at least one fragment or functional variant thereof that is capable of forming a dimeric protein, wherein the heterologous polypeptide is defined as being different from the interleukin 2 (moiety) of the first fusion polypeptide;

b. culturing transfected cells under conditions appropriate for expressing the nucleotide sequence or sequences coding for the first and second fusion polypeptides and association of two fusion polypeptides in vivo to form a heterodimeric protein;

c. recovering, and preferably purifying, the heterodimeric proteins formed.

Preferably, in the second fusion polypeptide, C4BPβ or said fragment is fused to the C-terminal end of the heterologous polypeptide.

The term “different” when referring to the heterologous polypeptide means a polypeptide which has a primary amino acid sequence that is different by at least one amino acid from the primary sequence of the interleukin 2 (moiety) of the first fusion polypeptide. Alternatively, the term “different” also covers heterologous polypeptides having the same primary sequence but having different post-translational modifications, for example in terms of acetylation, amidation, biotinylation, carboxylation, hydroxylation, methylation, phosphorylation or sulfatation, or by adding lipids (isoprenylation, palmitoylation and myristoylation), glucides (glycosylation) or polypeptides (ubiquitination).

In a preferred embodiment, the heterologous polypeptide is not IL2.

In a particular embodiment, the heterologous polypeptide may be selected from the group consisting of an auto-antigen, an antibody or antibody fragment including those targeting such auto-antigens, and a receptor, e.g. including the alpha chain of the IL2R, or a receptor ligand. Such constructs are particularly useful in treating autoimmune and/or inflammatory disorders. In such embodiment, the IL-2 moiety of the first fusion polypeptide is preferably an active variant that promotes Treg cell proliferation, survival, activation and/or function.

In another particular embodiment, the heterologous polypeptide may be selected from the group consisting of a tumor antigen, a microbial antigen, an antibody or antibody fragment including those targeting such antigens, or a receptor, including the alpha chain of the IL2R, or a receptor ligand. Such constructs are particularly useful in treating cancers. In this case, the IL-2 moiety of the first fusion polypeptide is preferably an active variant that promotes Teff cell proliferation, survival, activation and/or function.

Such heterodimer protein is also part of the invention.

In a particular embodiment, the host cell allows co-expression of the two fusion polypeptides, a first fusion polypeptide A comprising i) at least one interleukin 2 (IL2) moiety and ii) a beta chain of the C4b-binding protein (C4BPβ) or at least one fragment or functional variant thereof that is capable of forming a dimeric protein; and a second fusion polypeptide B, comprising i) at least one heterologous polypeptide and ii) a beta chain of the C4b-binding protein (C4BPβ) or at least one fragment or functional variant thereof that is capable of forming a dimeric protein, wherein the heterologous polypeptide is defined as being different from the interleukin 2 (moiety) of the first fusion polypeptide. In this particular embodiment, co-expression of the two fusion polypeptides can also allow the production of homodimers A-A and B-B and the production of heterodimers A-B.

It is also provided a recombinant eukaryotic cell allowing synthesis of a dimer or heterodimer protein as defined above, and obtainable by carrying out step a) of the production method defined above. Greater details for the production in host cells are described below.

Production Methods

The chimeric construct, which is in the form of a fusion protein, and the homo- or heterodimers can be produced by DNA recombinant technique in a suitable expression vector.

The expression vector is selected as a function of the host cell into which the construct is introduced. Preferably, the expression vector is selected from vectors that allow expression in eukaryotic cells, especially from chromosomal vectors or episomal vectors or virus derivatives, in particular vectors derived from plasmids, yeast chromosomes, or from viruses such as baculovirus, papovirus or SV40, retroviruses or combinations thereof, in particular phagemids and cosmids. In a particular embodiment, it is a vector allowing the expression of baculovirus, capable of infecting insect cells.

If necessary, the sequence coding for the fusion polypeptide also comprises, preferably in its 5′ portion, a sequence coding for a signal peptide for the secretion of fusion polypeptide. Conventionally, the sequence of a signal peptide is a sequence of 15 to 20 amino acids, rich in hydrophobic amino acids (Phe, Leu, lie, Met and Val).

The vector comprises all of the sequences necessary for the expression of the sequence coding for the fusion polypeptide. In particular, it comprises a suitable promoter, selected as a function of the host cell into which the construct is to be introduced.

Within the context of the invention, the term “host cell” means a cell capable of expressing a gene carried by a nucleic acid which is heterologous to the cell and which has been introduced into the genome of that cell by a transfection method.

Preferably, a host cell is a eukaryotic cell. A eukaryotic host cell is in particular selected from yeast cells such as S cerevisiae, filamentous fungus cells such as Aspergillus sp, insect cells such as the S2 cells of Drosophila or sf9 of Spodoptera, mammalian cells and plant cells. Mammalian cells which may in particular be cited are mammalian cell lines such as CHO, COS, HeLa, C127, 3T3, HepG2 or L(TK−) cells. In a preferred implementation, said host cells are selected from eukaryotic cell lines, preferably Sf9 insect cells. Methods for preparing recombinant dimeric proteins in sf9 insect cells are described in U.S. Pat. No. 7,884,190.

Any transfection method known to the skilled person for the production of cells expressing a heterologous nucleic acid may be used to carry out step a) of the method. Transfection methods are, for example, described in Sambrook et al, 2001, “Molecular Cloning: A Laboratory Manual”, 3rd edition, Cold Spring Harbor Laboratory Press, Cold Spring Harbor, N.Y.

Alternatively, the chimeric construct can be produced by chemical peptide synthesis. For instance, the protein can be produced by the parallel synthesis of shorter peptides that are subsequently assembled to yield the complete sequence of the protein with the correct disulfide bridge. A synthesis of IL-2 is illustrated for instance in Asahina et al., Angewandte Chemie International Edition, 2015, Vol. 54, Issue 28, 8226-8230, the disclosure of which being incorporated by reference herein.

In another embodiment, the chimeric protein may be expressed in vivo, after administering the subject with a nucleic acid encoding said chimeric protein. In a preferred embodiment, the nucleic acid is carried by a viral vector, such as an adeno-virus associated virus (AAV).

Formulations and Routes of Administration

It is also provided a pharmaceutical composition comprising a construct, a nucleic acid, a vector or a protein as described herein, preferably in association (e.g., in solution, suspension, or admixture) with a pharmaceutically acceptable vehicle, carrier or excipient.

Suitable excipients include any isotonic solution, saline solution, buffered solution, slow release formulation, etc. Liquid, lyophilized, or spray-dried compositions are known in the art and may be prepared as aqueous or nonaqueous solutions or suspensions. Preferably the pharmaceutical compositions comprise appropriate stabilizing agents, buffering agents, bulking agents, or combinations.

The pharmaceutical composition may further contain another active ingredient, or may be administered in combination with any other active ingredient.

The pharmaceutical composition may be administered using any convenient route, including parenteral, e.g. intradermal, subcutaneous, or intranasal route. The subcutaneous route is preferred. Oral, sublingual or buccal administrations are also encompassed.

An example of a formulation suitable for a subcutaneous injection is described in international patent application WO2017/068031.

Treatment of Auto-Immune and/or Inflammatory Disorders

The pharmaceutical compositions described herein are useful in methods for treating an auto-immune and/or inflammatory disorder, such as systemic lupus erythematous, type I diabetes, HCV-related vasculitis, uveitis, myositis, systemic vasculitis, psoriasis, allergy, asthma, Crohn's disease, multiple sclerosis, rheumatoid arthritis, atherosclerosis, autoimmune thyroid disease, auto-inflammatory diseases, neuro-degenerative diseases, including Alzheimer's disease and amyotrophic lateral sclerosis, acute and chronic graft-versus-host disease, spontaneous abortion and allograft rejection; solid organ transplantation rejection, vasculitis, inflammatory bowel disease (IBD), and allergic asthma; spondyloarthritis or ankylosing Spondylitis; Sjogren's syndrome, Systemic sclerosis, Alopecia aerate, or Ulcerative Colitis.

In a preferred embodiment, it is herein described a method of treatment of an auto-immune and/or inflammatory disorder, comprising administering the composition once or twice a week, or even once or twice a month, preferably by subcutaneous route. In one embodiment, a dosage of less than 30 MIU/day, preferably less than 20 MIU/day is preferred, advantageously less than 10 MIU/day, or between 1 MIU/day and 8 MIU/day. In another particular embodiment, a dose of between 1 and 5 MIU/day, preferably from 0.1 to 3.5 MIU/day is used

Generally speaking, doses that allow a 1.5, 2, 3, 4 or 5-fold increase of the number of Tregs are preferred The standard measure of an amount IL-2 is the International Unit (IU), which technically is not a fixed weight but the amount that produces a fixed biological effect in a specific cell proliferation assay, as determined by the World Health Organization (WHO). The reason is that i) the weight varies depending on the exact sequence of the molecule and its glycosylation profile, and ii) what matters is the activity, not the weight of the molecule.

The principle of the International Unit is precisely to provide a standard to which any IL-2 molecule can be compared (regardless of their source, or their sequence, including wild-type or active variant sequences).

In practice, the WHO provide ampoules containing an IL-2 molecule that has been calibrated and serves as the reference to determine the dosage of a given preparation of IL-2 (again regardless of the source or sequence of said IL-2) defined by its potency. For instance, to determine the dosage of a given preparation of IL-2, the biological activity of the candidate IL-2 preparation is measured in a standard cell proliferation assay using an IL-2 dependent cell line, such as CTLL-2, and compared with the biological activity of the standard. The cells are grown in the presence of different doses of the standard. A dose-response effect of IL-2 is established, where the dose of IL-2 is plotted on the X axis as IU and the measure of proliferation (pr) is on the Y axis. When one wants to determine the activity of any IL-2 product of unknown activity, the product is used to grow the IL-2 dependent cells and the proliferation is measured. The pr value is then plotted on the Y axis and from that value a line parallel to the X axis is drawn. From the point of intersection of this line with the dose response line, a line parallel to the Y axis is then drawn. Its intersection with the X axis provides the activity of the candidate IL-2 product in IU.

Any change of the WHO standard ampoules does not impact the International Unit nor the determination of a dosage of any IL-2 preparation.

The 1st standard (WHO international Standard coded 86/504, dated 1987) contained a purified glycosylated IL-2 derived from Jurkat cells and was arbitrarily assigned a potency of 100 IU/ampoule. As the stocks of the 1st international standard (IS) were running low, the WHO had to replace it. The WHO provided another calibrated IL-2 ampoule, this time produced using E. coli. The 2nd standard ampoules contained 210 IU of biological activity per ampoule. The change of standard ampoules does not mean that the IU changes. So, determining the dosage of a test IL-2 preparation will not vary whether one uses the 1st standard ampoule or the 2nd standard ampoule, or a subsequent standard ampoule, as a reference.

In one embodiment, a chronic administration is implemented, e.g. comprising administration once every 3 days to once every three months. Such sequences of administration may be repeated if needed.

In another embodiment, the IL-2 is given every other day for 1 to 2 weeks, in cycles that can be repeated after break of administration that can last from 3 days to 3 months, preferably from one to 4 weeks.

In another embodiment, the treatment may comprise a first course that is also designated as an induction course, and a second course, that is maintenance course.

In a particular embodiment, the treatment may comprise at least a first course wherein the pharmaceutical composition is administered once per day during at least about 2 or 3 consecutive days, preferably during 3 to 7, still preferably during 4 to 5 consecutive days, preferably followed by a maintenance dose, e.g. after about six days or about 1 to about 4 weeks.

The maintenance dose may be typically administered during at least one month, preferably at least about 3 months, still preferably at least about 6 months. In a preferred embodiment, the maintenance dose is administered between about 3 months and about 12 months, preferably between about 6 months and about 12 months.

In a preferred embodiment, the maintenance treatment consists of an administration of the pharmaceutical composition once or twice a week, or every one or two weeks, or once a month.

In a preferred embodiment, the maintenance treatment consists of an administration of interleukin-2 once or twice a week, every one or two weeks, or once a month during a period of at least one month, preferably from about 3 months to about 12 months.

Preferably the maintenance dosage is substantially the same as the first course dosage, or it can be a lower or higher dosage.

Treatment of Cancers

The pharmaceutical compositions described herein are useful in methods for treating a cancer. In some embodiments, the subject is suffering from locally advanced or metastatic cancer. In some embodiments, the cancer is a solid tumor. In some embodiments, the cancer is colon cancer, lung cancer, ovarian cancer, gastric cancer, bladder cancer, pancreatic cancer, endometrial cancer, breast cancer, kidney cancer, esophageal cancer, or prostate cancer.

In one embodiment, a dosage of less than 30 MIU/day, preferably less than 20 MIU/day is preferred, advantageously less than 10 MIU/day, or between 3 MIU/day and 5 MIU/day.

In other embodiments, 400,000-750,000 IU/kg or 550,000-750,000 IU/kg, preferably 600,000-700,000 IU/kg, IL2 is administered. The dosage may be similar to, but is expected to be less than, that prescribed for PROLEUKIN®.

The compositions can be administered once from one or more times per day to once or more times per week; including once every other day. The skilled artisan will appreciate that certain factors may influence the dosage and timing required to effectively treat a subject, including but not limited to the severity of the disease, previous treatments, the general health and/or age of the subject, and other diseases present. Moreover, treatment of a subject can include a single treatment or, can include a series of treatments.

The examples of protocols described above in connection with auto-immune and/or inflammatory disorders may be applied identically or similarly for use in treating a cancer. Alternatively, in another example, the compositions may be administered every 8 hours for five days, followed by a rest period of 2 to 14 days, e.g., 9 days, followed by an additional five days of administration every 8 hours. In some embodiments, administration is 3 doses administered every 4 days.

The Examples and Figures illustrate the invention without limiting its scope.

### EXAMPLES

### Example 1: Production and Characterization of the IL-2/C4BP Fusion Proteins

Lentiviral vectors were used for production of IL-2 fusion proteins. Briefly, human IL-2 (Hi2, SEQ ID NO:1), human IL-2 fused to the C-terminal region of C4BPß (Hi2cb, SEQ ID NO:6) or the same molecule with a mutated IL-2 (N88R variant; Hi2mcb SEQ ID NO: 7) were integrated in a lentiviral plasmid under the spleen focus-forming virus (SFFV) promoter. HEK 293T cells were transfected at 70% confluence with lentiviral plasmid using polyethylenimine (PEI) and cultured for 48H in a serum-free medium. Supernatants were then filtered and concentrated by ultracentrifugation and resuspended in appropriate buffer before conservation at −80° C. To obtain stable transfected cells, HEK 293T cells were infected with lentivirus at different multiplicity of infection (MOI) and transduction efficiencies were evaluated by flow cytometry using the Green-fluorescent protein (GFP) produced as selection marker. Cells with at least 50% of transduction efficiency and 80% viability were put in cultured with complete medium for a week before cell sorting of GFP+ cells to ensure almost 100% of cells producing IL-2 fusion proteins.

To perform in vitro evaluation of the functional design of the constructs (Western blot, human whole blood STAT5 phosphorylation), stable cell lines were put in culture for 48H in a serum-free medium and supernatants were harvested, filtered, concentrated and purified by chromatography before utilization.

Recombinant adeno-associated viruses (AAV) were produced by transfection of an AAV2/8 vector (virus containing the genome of serotype 2 packaged in the capsid from serotype 8 AAV) with the same transgenes of interest and an auxiliary plasmid in HEK 293T cells. The AAV vectors were extracted from the culture supernatants, which were clarified by centrifugation and viruses were purified on a cesium chloride gradient and dialyzed.

Hi2cb and Hi2mcb were then characterized by Western blot using either a primary anti-human IL-2 antibody or a primary anti-human C4bpß antibody. Under reduced conditions, Hi2cb and Hi2mcb are detected at a molecular weight of approximately 23 kDa corresponding to monomers. Under normal conditions, two bands were detected with a major signal at a molecular weight of approximately 46 kDa, which corresponds to dimers. A second weak signal corresponds to monomers at approximately 23 kDa.

### Example 2: Treg Selectivity of the Fusion Proteins in Human Whole Blood pSTAT5 Response

Immunophenotype. Human whole blood from healthy adults was collected with informed consent. The effect of Hi2, Hi2cb, or Hi2mcb on the STAT5 phosphorylation (pSTAT5) was assessed in human CD4+ regulatory T cells (Treg; CD4+Foxp3+CD127lo/−), CD4+ conventional T cells (Tconv; CD4+Foxp3-), and CD8+ T cells using flow cytometry. Ten-fold dilution of Hi2, Hi2cb and Hi2mcb were mixed with 100 μl of whole blood for 15 min at 37° C. before pSTAT5 staining.

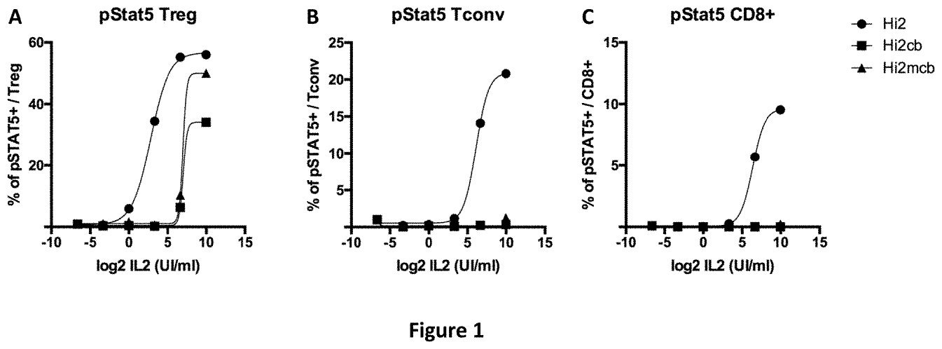

FIG. 1 shows the dose-response of pSTAT5 induction on different populations of interest.

Hi2 is able to induce phosphorylation of STAT5 in Treg, Tconv and CD8 T cells. Both IL-2-C4bpß proteins are also able to induce STAT5 phosphorylation on Treg while not able on Tconv and CD8 T cells, showing the capacity of fusion proteins to selectively target Tregs in a larger therapeutic window.

### Example 3: In Vivo Evaluation of IL-2-C4 bpß-Coding AAV on T Cells and Glomerular Filtration

Mice. Six to eight weeks old C57BL/6 (Jrj) female mice were injected by intraperitoneal route with 10¹¹ viral genomes (vg) of AAV coding for Hi2, Hi2cb or Hi2mcb.

Immunophenotype. Blood samples were collected once a week, in heparin tubes to avoid clot formation. After hemolysis, immune cells were stained and analyzed by flow cytometry to determine percentages of Treg (CD4+CD25+Foxp3+), Teff (CD4+CD25+Foxp3-), CD8+ Treg (CD8+CD25+Foxp3+) and CD8+ Teff (CD8+CD25+Foxp3-).

Dosage. Blood and urine were collected at different time points to dose IL-2 using a human IL-2 uncoated ELISA kit (ThermoFisher).

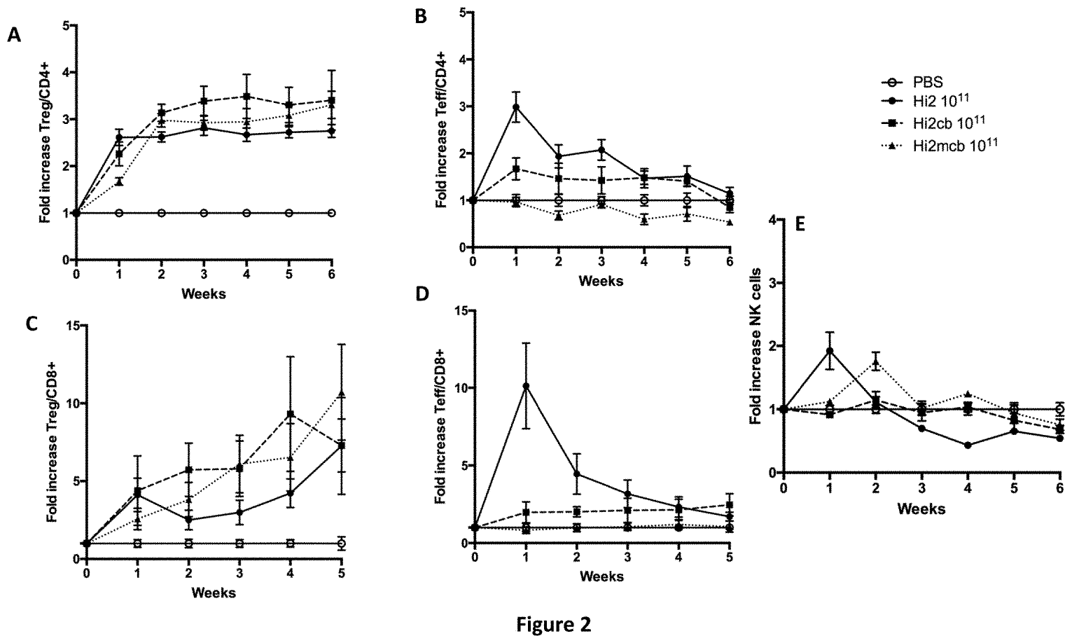

FIG. 2 shows kinetic curves of the fold increase of the four different T cell compartments, compared to control. Regarding regulatory T cells, the 3 proteins induce a great and comparable increase of Tregs (FIG. 2A) and CD8+ Tregs (FIG. 2C), with even a slight better increase with fusion proteins, with a higher plateau compared to Hi2. Whereas Hi2 induces a huge peak of increase of Teffs (3-fold, FIG. 2B) and CD8+ Teffs (10-fold, FIG. 2D) and an expansion of NK cells after one week (2-fold, FIG. 2E), Hi2cb seems to prevent it with a slight increase of around 1.5-fold for both CD4+ and CD8+ T cells and a complete control of the expansion of NK cells. The mutated form of the fusion protein, Hi2mcb, demonstrates a complete control of the effector compartment in both CD4+ and CD8+T cells, as well as a control of the NK cells despite a transitory increase, showing the capacity of this mutation to favor Treg selectivity.

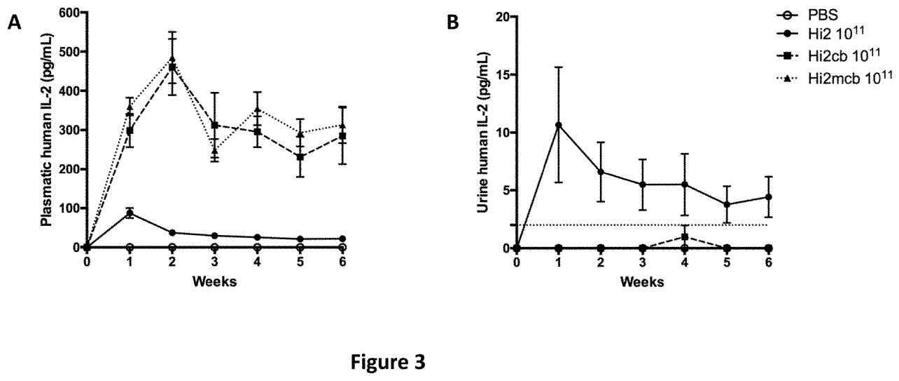

FIG. 3 shows the kinetic of plasma (A) and urinary (B) human IL-2 over the time. Important differences between Hi2 and IL-2-C4bpß fusion proteins kinetics in both plasma and urine are observed. Hi2 has a peak after 1 week with a sustained plateau around 20 pg/mL (FIG. 3A). To compare, fusion proteins have a later peak at week 2 with a very high and sustains plateau around 300 pg/mL. In urine, human IL-2 is only detected after hIL-2 coding-AAV whereas no human IL-2 is detected in urine after IL-2-C4bpß-coding AAV administration. These results highlight the capacity of these fusion proteins not to be filtered by kidney, suggesting an increase of the half-life of these IL-2-C4bpß fusion proteins in the plasma.

### Example 4: Toxicity of Hi2, Hi2cb or Hi2mcb after a High-Dose Administration of AAV

Mice. Six to eight weeks old C57BL/6 (Jrj) female mice were injected by intraperitoneal route with 10¹² vg of AAV coding for Hi2, Hi2cb or Hi2mcb.

Immunophenotype. Blood samples were collected once a week, in heparin tubes to avoid clot formation. After hemolysis, immune cells were stained and analyzed by flow cytometry to determine percentages of Treg (CD4+CD25+Foxp3+), Teff (CD4+CD25+Foxp3-), CD8+ Treg (CD8+CD25+Foxp3+) and CD8+ Teff (CD8+CD25+Foxp3-).

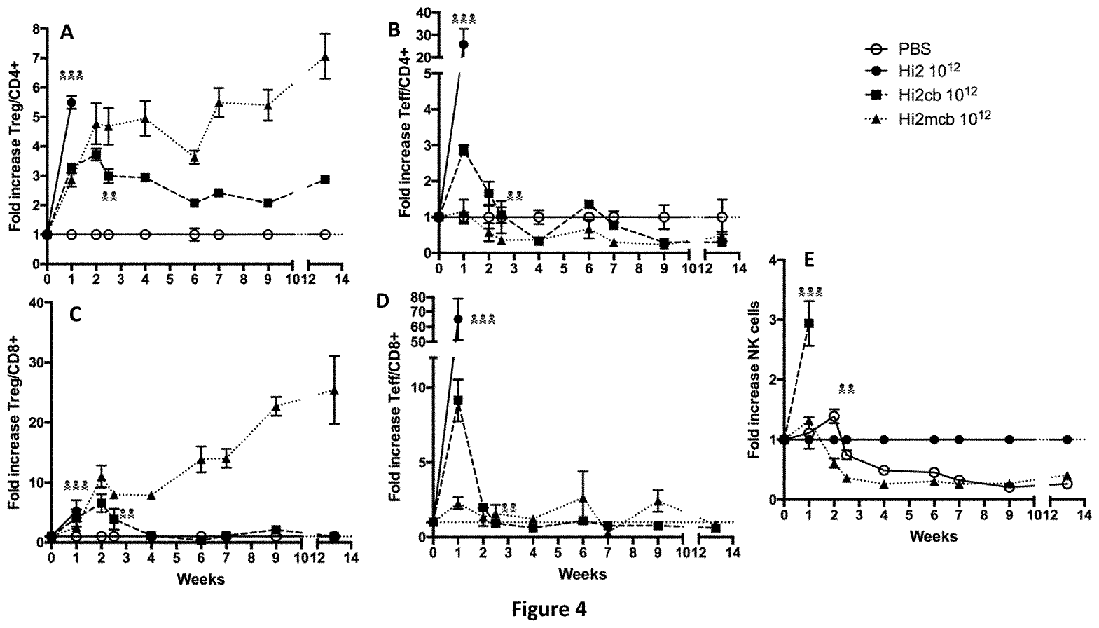

FIGS. 4A-4E show kinetic curves of the fold increase of the four different T cell compartments, compared to control. Effect of the 3 constructions on effector and regulatory T cells are quite similar to those with a lower dose. Briefly, Hi2mcb increases both CD4+ and CD8+ Tregs while perfectly controls the increase in both effector levels and NK cells. This regulatory T cells selectivity is very likely the reason for the good safety profile.

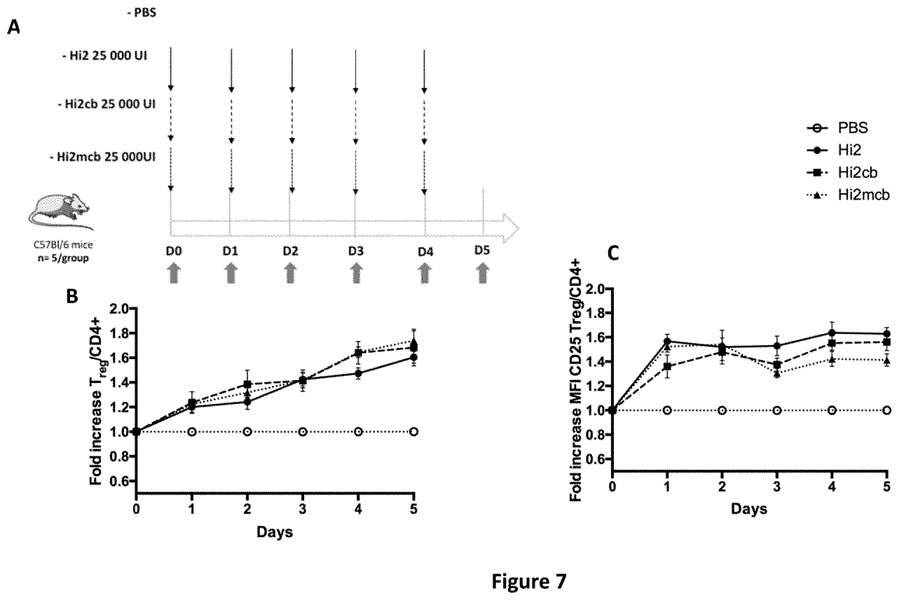

Kinetics after Hi2cb-coding AAV demonstrate the capacity to control only partially the increase in CD4+(3-fold increase at peak, FIG. 4B) and CD8+(9-fold increase at peak, FIG. 4D) effector compartments. However, both Treg compartments were increased after 2 weeks, reaching almost 4-fold increase for Tregs (FIG. 4A) and 7-fold increase for CD8+ Tregs (FIG. 4C). Finally, in the group of mice treated with Hi2 all of them died within 1 week while there was a great CD4+ and CD8+ Tregs increase (FIG. 4A,C), and a dramatic expansion of Teffs (25-fold increase, FIG. 4B), CD8+ Teffs (62-fold increase, FIG. 4D) and NK cells (3-fold increase, FIG. 4E) that probably cause a profound imbalance in the immune homeostasis leading to a rapid death.

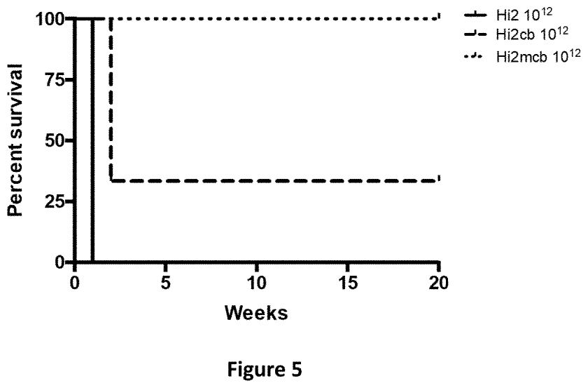

FIG. 5 shows the Kaplan-Meier curve of mice after high-dose administration of AAV. The toxicity evaluation of high-doses of the 3 proteins demonstrates a very good safety profile for IL-2-C4bpß fusion proteins compared to classical Hi2 (FIG. 5). Indeed, all mice injected with Hi2 died within 7 days. A delay of mortality was observed with Hi2cb since 2 out of 3 mice died at day 17 only. Finally, all mice treated with Hi2mcb are still alive 20 weeks after the beginning of the experiment highlighting the complete safety of the fusion protein.

### Example 5: Therapeutic Efficacy of Fusion Proteins in a Model of Experimental Autoimmune Encephalomyelitis

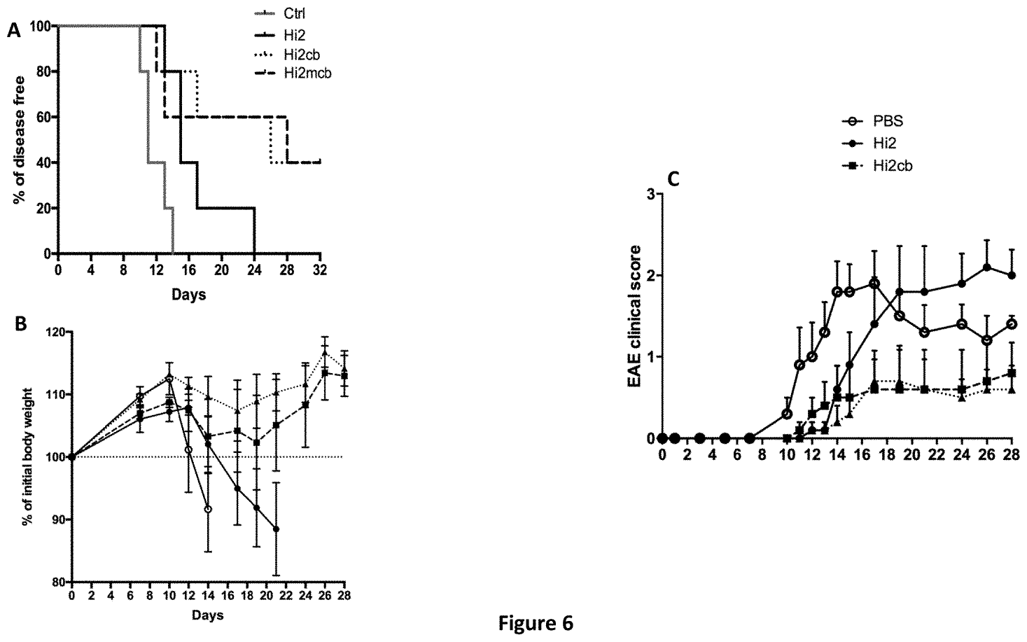

Mice. Six to eight weeks old C57BL/6 (Jrj) female mice were injected by intraperitoneal route with 10¹¹ vg of AAV coding for Hi2, Hi2cb or Hi2mcb seven days before experimental autoimmune encephalomyelitis model (EAE) induction to ensure Treg expansion at the initiation of the disease. Preventive treatment with every IL-2 based molecules delay the clinical onset (FIG. 6A). However, 3 to 5 days after disease onset, mice treated with standard IL2 (black dots) showed a similar kinetic of the clinical symptoms when compared to control mice (clear dots, FIG. 6C). This result is confirmed and observed in all analysed parameters, with an important weight loss (FIG. 6B) and 100% of mice developing clinical symptoms (FIG. 6A). Conversely, mice treated with both fusion proteins showed the same delay of onset but with a control of clinical symptoms severity (black squares and triangles respectively), a total control of weight loss (FIG. 6B) as well as 40% of complete disease prevention meaning mice without any clinical symptom (FIG. 6A).

### Example 6: Production and Evaluation of Fusion Proteins

6.1. Production of Fusion Proteins

Purified fusion proteins were obtained from stable cells line production. In details, HEK 293T cells were put in culture and supernatants were collected, and purified by a size-exclusion chromatography usin an AKTA™ system. ELISA and Western blot were performed to collect the positive fractions. Finally, collected samples were passed through an anion exchange chromatography.

6.2. Immunophenotyping

Mice. Six to eight weeks old C57BL/6 (Jrj) female mice were injected by subcutaneous route with Hi2, Hi2cb or Hi2mcb (25 000 UI) every day for five days.

Immunophenotype. Blood samples were collected once a day before injection, in heparin tubes to avoid clot formation. After hemolysis, immune cells were stained and analyzed by flow cytometry to determine percentages of Treg (CD4+CD25+Foxp3+) and CD25 mean fluorescence intensity on Treg.

FIG. 7A shows the experimental administration schedule based on one injection of 25 000 International Units of proteins every day during five days. Immunophenotyping were performed every single day for five days just before injection.

FIGS. 7B-7C show kinetic curves of the fold increase T cell compartments and NK cells, compared to control. Regulatory T cells expansion are quite similar between Hi2 and fusion proteins as well as CD25 MFI on Tregs with 1.6-fold increase.

6.3. Dosage

Blood and urine were collected at different time points to dose IL-2 using a human IL-2 uncoated ELISA kit (ThermoFisher).

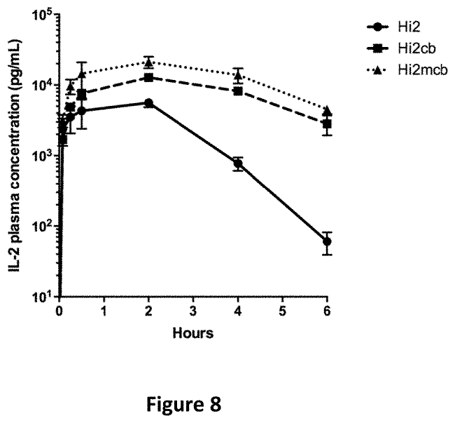

Six to eight weeks old C57BL/6 (Jrj) female mice received a single administration of Hi2, Hi2cb, or Hi2mcb supernatant proteins by subcutaneous route and plasmatic concentrations of hIL-2 were determined at different time points. Major pharmacokinetics differences are observed between Hi2 and fusions proteins (FIG. 8). Indeed, fusion proteins are able to stay longer in the plasmatic compartment with reduced elimination compare to Hi2. These results highlight the fact that these fusion proteins have increased half-life with reduced clearance elimination.

1. A chimeric construct comprising i) at least one interleukin 2 (IL2) moiety and ii) a beta chain of the C4b-binding protein (C4BPβ) or at least one fragment or functional variant thereof that is capable of forming a dimeric protein.

2. The chimeric construct of claim 1, wherein the fragment of C4BPβ comprises, or consists of, amino acid residues 194 to 252 of C4BPβ or a longer fragment of C4BPβ that extends at the N-term up to at most amino acid 135.

3. The chimeric construct of claim 1 or 2, comprising a functional variant of C4BPβ which comprises
a) a modified sequence of the fragment of C4BPβ, wherein less than 25 percent of the amino acids of the fragment, preferably less than 10 percent, have been cut out or replaced, in which the cysteines located in positions 202 and 216 as well as at least 3 amino acids upstream and downstream of each cysteine have been conserved; or
b) a modified sequence of the fragment of the C4BPβ, wherein a cysteine responsible for dimerization is substituted with an amino acid, preferably selected from alanine, valine, phenylalanine, proline, methionine, isoleucine, leucine and tryptophan, and another amino acid of the fragment is substituted with a cysteine; or
c) a sequence of the fragment of C4BPβ modified by insertion of a sequence which is heterologous to the beta chain, between the cysteines responsible for dimerization; or
d) a sequence of the fragment of C4BPβ modified by cutting out amino acids between the cysteines responsible for dimerization.

4. The chimeric construct according to any of claims 1 to 3, wherein said IL-2 moiety is human IL-2 or homologous variant thereof, wherein the variant has at least 85% amino acid identity with human wild-type IL-2, preferably wherein the variant is an active analogue of human IL-2 which has at least 90% amino acid identity with human wild-type IL-2, wherein said IL-2 moiety is preferably an IL2 mutein that comprises a substitution at position N88 of SEQ ID NO: 2, still preferably substitution N88R.

5. The chimeric construct according to any of claims 1 to 4, wherein the IL2 moiety and C4BPβ or said fragment thereof are fused in frame or through an amino acid linker, preferably a polyG linker.

6. The chimeric construct according to any of claims 1 to 5, wherein the IL2 moiety is fused at the N-terminus of C4BPβ or said fragment thereof, preferably wherein the chimeric protein is a fusion protein wherein one IL2 moiety is fused at the N-term of C4BPβ or of said fragment thereof, and another IL2 moiety is fused at the C-term of C4BPβ or of said fragment thereof.

7. A homodimer protein comprising two fusion polypeptides, each consisting of the chimeric product of any of claims 1 to 6.

8. A method for producing a recombinant dimer protein as defined in claim 6, comprising:
a) transfecting a host cell with a vector allowing expression of a nucleotide sequence coding for a fusion polypeptide that is the chimeric construct as defined in any of claims 1 to 6;
b) culturing transfected cell under conditions which are suitable for expressing the nucleotide sequence coding for the fusion polypeptide and the covalent association of two fusion polypeptides in vivo to form a dimeric protein;
c) recovering the dimeric protein formed.

9. A heterodimer protein comprising two fusion polypeptides, wherein a first fusion polypeptide consists of the chimeric product of any of claims 1 to 6 and the second comprises i) at least one heterologous polypeptide and ii) a beta chain of the C4b-binding protein (C4BPβ) or at least one fragment or functional variant thereof that is capable of forming a dimeric protein, wherein the heterologous polypeptide is different from the IL-2 moiety of the first fusion polypeptide, preferably wherein the heterologous polypeptide is an auto-antigen or a tumor antigen.

10. A method for producing a recombinant heterodimer protein as defined in claim 9, said method comprising:
a. transfecting a host cell with one or more vectors to allow the expression of one or more nucleotide sequences coding for:
i. a first fusion polypeptide that is the chimeric construct as defined in any of claims 1 to 6; and
ii. a second fusion polypeptide, comprising i) at least one heterologous polypeptide and ii) a beta chain of the C4b-binding protein (C4BPβ) or at least one fragment or functional variant thereof that is capable of forming a dimeric protein, wherein the heterologous polypeptide is different from the interleukin 2 moiety of the first fusion polypeptide;
b. culturing transfected cells under conditions appropriate for expressing the nucleotide sequence or sequences coding for the first and second fusion polypeptides and association of two fusion polypeptides in vivo to form a heterodimeric protein;
c. recovering the heterodimer protein formed.

11. A nucleic acid encoding the chimeric construct of any of claims 1 to 6.

12. A vector comprising the nucleic acid of claim 11.

13. A host cell comprising the nucleic acid of claim 11 or the vector of claim 12.

14. The homodimer protein of claim 7 or the heterodimer protein of claim 9, for use in treating an inflammatory and/or autoimmune disorder in a subject.

15. The homodimer protein of claim 7 or the heterodimer protein of claim 9, for use in treating a cancer in a subject.
